(19) United States
(12) Patent Application Publication
(10) Pub. No.: US 2014/0045252 A1
(43) Pub. Date: Feb. 13, 2014

(54) CELL CULTIVATION CONTAINER AND CELL CULTURING APPARATUS

(71) Applicants: Ryota Nakajima, Tokyo (JP); Toyoshige Kobayashi, Tokyo (JP); Shizu Matsuoka, Tokyo (JP); Takayuki Nozaki, Tokyo (JP)

(72) Inventors: Ryota Nakajima, Tokyo (JP); Toyoshige Kobayashi, Tokyo (JP); Shizu Matsuoka, Tokyo (JP); Takayuki Nozaki, Tokyo (JP)

(73) Assignee: HITACHI, LTD., Tokyo (JP)

(21) Appl. No.: 14/113,857

(22) PCT Filed: Apr. 2, 2012
(86) PCT No.: PCT/JP2012/058975

§ 371 Date: Oct. 25, 2013

(30) Foreign Application Priority Data
Apr. 28, 2011 (JP) 2011-101057

(51) Int. Cl. C12M 1/42

(52) U.S. Cl. CPC C12M 35/02
USPC 435/288.7; 435/289.1

(57) ABSTRACT

Provided is a cell cultivation container equipped with electrodes, which enables automatic culturing, observation of automatically cultured cells, and measurement of electrical resistances. A circular electrode (10) is arranged on a base section or a side surface of a frame body (2) of a cell cultivation container, in such a manner that allows observation of cells. Additionally, a rod-shaped electrode (11) is arranged on a lid section (3) of the cell cultivation container, in such a manner that allows observation of cells. In an alternative configuration, the cell cultivation container has a flow channel (8) made of an electrically conductive material. An alternating current generator (13) is connected between the electrode (10) and the electrode (11), and the transepithelial electrical resistances of cells are measured during culturing. This configuration enables automatic culturing, observation of automatically cultured cells, and measurement of electrical resistances.

### TECHNICAL FIELD

The present invention relates to a cell culture container that realizes a non-invasive quality evaluation of a cell, and a cell culturing apparatus using the cell culture container.

### BACKGROUND ART

In a regenerative medicine for curing disease by using own cell or a cell of other person, a cell extracted from a living body is often cultured to increase its number or to form a tissue into an appropriate form, and then, used for a treatment. The cell used for the treatment has to be cultured in a cell culturing clean room called a cell processing center (CPC) in accordance with GMP (Good Manufacturing Practice). The problems are such that the preparation of a cell for one patient takes much labor and cost since the cell is manually cultured by a technical expert, and that there is a risk of biological contamination caused by a manual operation.

As a method of solving these problems, a device that automates a cell culturing process by using a closed system described in Patent Literature 1 has been developed. This device attains the automation of the cell culturing process and the reduction in the risk of the biological contamination by the use of the closed culture container that does not need an operation of opening and closing a lid of the culture container.

On the other hand, an invasive process such as a histological analysis is popular for a quality evaluation of a cell or a tissue after the culture, and it is difficult to evaluate the cell itself used for the treatment.

As a method of solving this problem, a method of non-invasively evaluating a function of a cell sheet by a measurement of transepithelial electrical resistance by utilizing a phenomenon in which an occluding junction is formed between cells in an epithelial cell sheet such as a corneal epithelial cell sheet (see Patent Literature 2, and Non-Patent Literature 1). A transepithelial electrical resistance measuring apparatus required for this method has already been commercially available from WPI (World Precision Instrument) Inc. and nanoAnalytics GmbH.

### CITATION LIST

### Patent Literature

PTL 1: Japanese Patent Application Laid-Open No. 2006-149237

PTL 2: Japanese Patent Application Laid-Open No. 2009-27928

### Non-Patent Literature

Non-Patent Literature 1: J. Wegener et al., “Automated multi-well device to measure transepithelial electrical resistances under physiological conditions”, BioTechniques, Vol. 37, No. 4 (2004), pp. 590

### SUMMARY OF INVENTION

### Technical Problem

However, the existing transepithelial electrical resistance measuring apparatus are not adapted to the culturing apparatus with closed system described in Patent Literature 1, and have a problem of being unable to measure the transepithelial resistance in real time with the closed space being kept during the automatic culture. The commercially available product or the electrode shape and electrode arrangement described in Patent Literature 2 entail a problem of being difficult to do an observation of a cell that has to be executed during the automatic culture. The commercially available product or the electrode shape and electrode arrangement described in Patent Literature 2 also entail a problem of being unable to culture a cell, such as nutritive cell, just below the culture face on a bottom of a lower layer of a culture layer, particularly the face close to an upper layer.

An object of the present invention is to provide a cell culture container that can solve all of these problems, and can realize a real-time cell observation and real-time measurement of transepithelial electrical resistance, and a cell culturing apparatus using the cell culture container.

### Solution to Problem

In order to attain the foregoing problem, the present invention provides a cell culture container for holding and culturing a cell, the cell culture container including: a frame body that holds culture liquid for culturing the cell; a lid that is detachably mounted on the frame body; a first electrode mounted on a bottom or on a side face of the frame body and having a shape enabling a cell observation; and a second electrode mounted on the lid and having a shape enabling a cell observation.

In order to attain the foregoing problem, the present invention also provides a cell culturing apparatus that measures an electric resistance of a cell in a cell culture container, the cell culturing apparatus including: a closed culture container for culturing a cell; a constant temperature reservoir for culturing a cell in which the closed culture container is placed; a control device that controls a culture environment in the closed culture container; and an AC voltage generating device, wherein the closed culture container includes a frame body that holds culture liquid for culturing the cell; a lid that is detachably mounted on the frame body; a first electrode mounted on a bottom or on a side face of the frame body and having a shape enabling a cell observation; and a second electrode mounted on the lid and having a shape enabling a cell observation, and the AC voltage generating device applies an AC voltage between the first electrode and the second electrode during the culture of the cell in the closed culture container.

### Advantageous Effects of Invention

A cell culture container according to the present invention can realize a real-time measurement of transepithelial electrical resistance of a cell and real-time cell observation in a state in which the cell is cultured with a closed space being kept during an automatic culture, whereby a safe and secure patient care can be realized after a quality of a cell or a tissue itself, which is to be transplanted, is evaluated.

### BRIEF DESCRIPTION OF DRAWINGS

FIG. 1 is a view illustrating a cross-section of a cell culture container on which an electrode is not arranged according to a first example.

FIG. 2 is a view illustrating one example of a configuration of the cell culture container on which an electrode is arranged according to the first example.

FIG. 3 is a view illustrating another example of a configuration of the cell culture container on which an electrode is arranged according to the first example.

FIG. 4 is a view illustrating another example of a configuration of the cell culture container on which an electrode is arranged according to the first example.

FIG. 5 is a view illustrating another example of a configuration of the cell culture container on which an electrode is arranged according to the first example.

FIG. 6 is a view illustrating another example of a configuration of the cell culture container on which an electrode is arranged according to the first example.

FIG. 7 is a view illustrating another example of a configuration of the cell culture container on which an electrode is arranged according to the first example.

FIG. 8 is a view illustrating another example of a configuration of the cell culture container on which an electrode is arranged according to the first example.

FIG. 9 is a view illustrating another example of a configuration of the cell culture container on which an electrode is arranged according to the first example.

FIG. 10 is a view illustrating an electric resistance when a corneal epithelial cell of a rabbit is cultured according to the first example.

FIG. 11 is a view illustrating a culture continuation determination program according to the first example.

FIG. 12 is a view illustrating a display screen of the culture continuation determination program according to the first example.

### DESCRIPTION OF EMBODIMENTS

Various examples of the present invention will be described below with reference to the accompanying drawings. It should be noted that these examples have been presented by way of example only, and are not intended to limit the technical scope of the invention. The same reference numerals are given to the same components in the drawings.

### EXAMPLE 1

The first example will be described with reference to the drawings.

### <Configuration of Cell Culture Container>

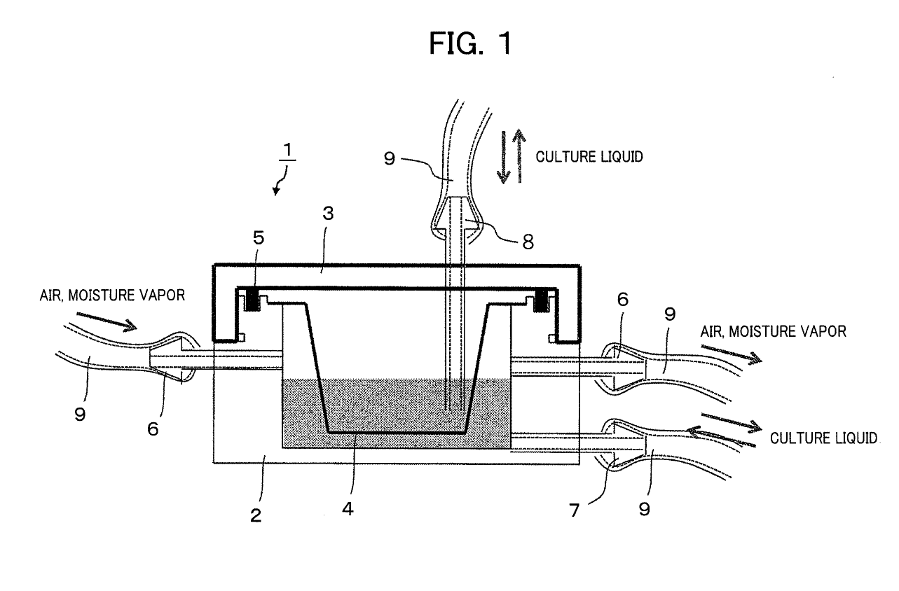

FIG. 1 is a view illustrating one example of a cross-sectional structure of a cell culture container according to the first example. The cell culture container 1 is a square container, and is made of plastics having plasticity and rigidity, such as polycarbonate, polystyrene, or polypropylene. A frame body 2 and a lid 3, which form the cell container 1, is formed by injection molding, and an insert container 4 can be inserted therein. A commercially available insert container can be used as the insert container 4. Usable containers are not limited, and include the one manufactured by BD Company, the one manufactured by Corning Incorporated, and the one manufactured by Greiner-Bio-One Co., Ltd. An elastic member 5 such as an O-ring is mounted to the lid 3 or the frame body 2, and with this elastic member 5, gas or particles containing bacteria do not enter from the outside. The lid 3 can be fixed to the frame body 2 by the threaded engagement of screws formed on the lid 3 and the frame body 2, but the fixing method is not limited thereto.

A pair of flow channels 6, which has a connection projection structure for injecting and exhausting air and moisture vapor on one end, is provided on the frame body 2. The position of the flow channel 6 on the frame body 2 has to be changed according to the volume of culture liquid injected into the container, but the position is only above the level of the injected culture liquid. The frame body 2 is also provided with a flow channel 7 having a projecting structure for injecting and exhausting the culture liquid on one end. The flow channel 7 is desirably mounted such that the bottom face of the frame body 2 and the lowermost part of the inner diameter of the flow channel 7 have the same height. This structure can allow the culture liquid to be efficiently injected and exhausted. In order to completely exchange the culture liquid, the frame body 2 may be inclined, according to need.

The lid 3 is provided with a flow channel 8 having a connection projecting structure for injecting and exhausting the culture liquid into and from the insert container 4 on its one end. The flow channel 8 is arranged not to interfere the observation of the cell. The arrangement not to interfere the observation means that the flow channel 8 has a shape not hindering an optical axis of a microscope, and the flow channel 8 is arranged on the position not hindering the optical axis of the microscope, when the inside of the cell culture container is observed by use of the microscope, for example. The flow channel 8 preferably has a length not touching the bottom surface of the insert container 4. A tube 9 having an inner diameter matching the size of the projecting structure of each flow channel and made of an elastic member such as silicon can be connected to the flow channels 6, 7, and 8. The tube 9 is needed for the connection with an automated culturing apparatus.

The flow channel for injecting and exhausting the culture liquid is provided for each of the frame body 2 and the lid 3. However, when an injection port and an exhaust port are separated, another flow channel is provided to each of the frame body and the lid to form a pair of flow channels.

### <Shape and Arrangement of Electrode>

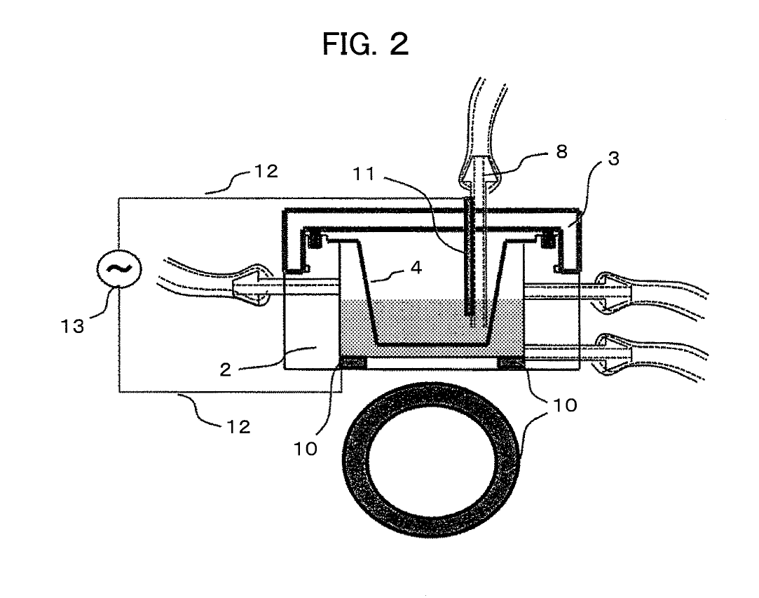

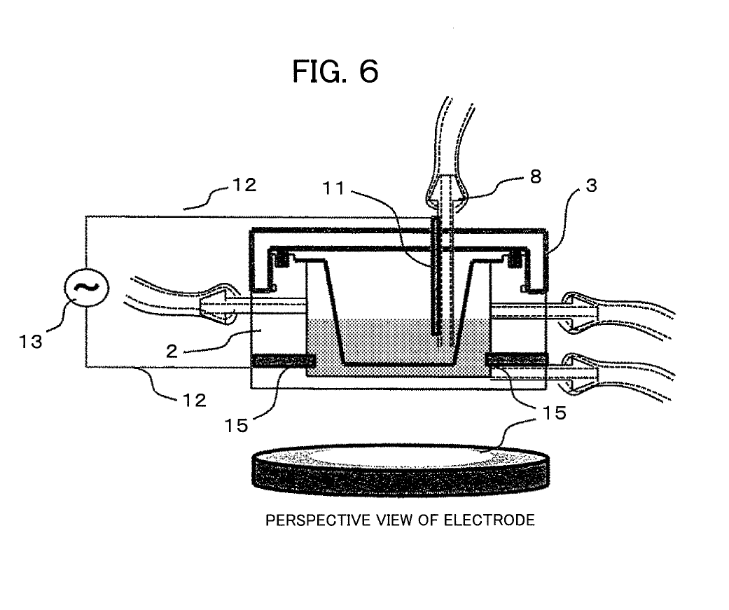

FIGS. 2 to 8 are views illustrating the structure in which electrodes with various shapes are arranged on the cell culturing apparatus according to the example. FIGS. 2 to 4 illustrate that the electrode is arranged on the bottom surface of the frame body, while FIGS. 6 to 8 illustrate that the electrode is arranged on the side face of the frame body.

In FIG. 2, a circular electrode 10 illustrated in a top view on the lower part of FIG. 2 is arranged on the bottom, which is the bottom surface serving as a culture holding surface, of the frame body 2, and a rod electrode 11 is arranged on the lid 3 along the flow channel 8 that penetrates through the lid 3. An electric wire 12 from the electrode 11 is arranged outside the frame body, and connected to an AC voltage generating device 13 that can measure an electric resistance. These electrodes have a shape enabling the observation of the cell with no trouble for the cell observation, and are arranged on the position not hindering the cell observation. The shape is referred to as the shape enabling the cell observation below, and this term means the shape and position that do not interfere with the optical axis of the microscope during the observation with the microscope in the cell culture container, for example. The electrodes 10 and 11 form an electrode unit in the cell culture container 1. The AC voltage generating device 13 and the cell culture container may be connected such that the electric wire 12 preliminarily connected to the AC voltage generating device 13 is connected to the electrode unit in the cell culture container, or the electric wire 12 preliminarily connected to the electrode unit in the cell culture container 1 is connected to the AC voltage generating device 13. In the present specification, the electrode 10 and the electrode 11 may sometimes be referred to as a first electrode and a second electrode.

The shape and arrangement of the electrode described above enable the cell adhesion onto the bottom of the frame body 2 and the observation of the cell on the bottom of the frame body 2 and the bottom of the insert container 4. The reason why the AC voltage is used for the measurement of the transepithelial electrical resistance of the cell is to prevent the cell and the tissue from being damaged. The commercially available products from WPI Inc. and nanoAnalytics GmbH also use the AC voltage.

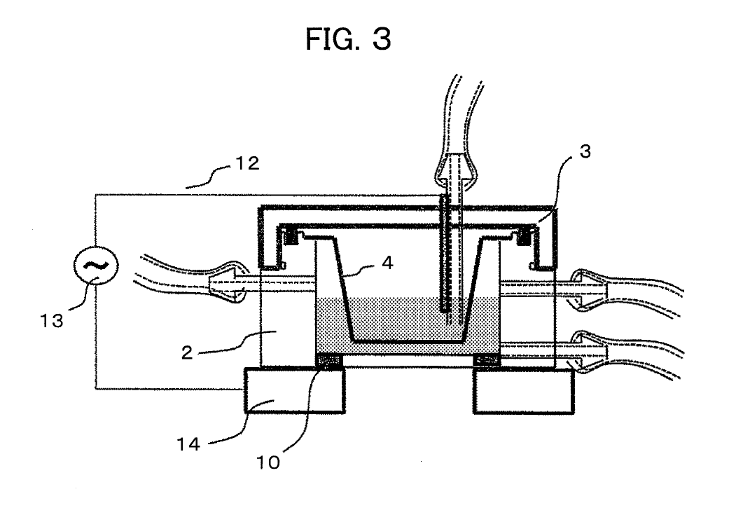

FIGS. 3 to 5 are views illustrating the configuration of modifications of the example of the cell culture container on which the electrode described with reference to FIG. 2 is arranged. In FIG. 3, the electric wire is not directly connected to the electrode 10 on the bottom of the frame body 2, but a cell culture container table 14 that can be energized is provided on the bottom of the frame body 2. With this structure, only when the cell culture container 1 is placed on the cell culture container table 14, the electric resistance can be measured as for the electrode 10 on the bottom 2. Specifically, an electrode is provided on the position of the cell culture container table 14 that is in contact with the electrode 10 of the cell culture container 1 when the cell culture container 1 is placed, and the electric wire 12 connected to the table 14 is connected to the AC voltage generating device 13 having a function of measuring the electric resistance. The AC voltage generating device 13 can be configured to calculate the electric resistance of various cells such as an epithelial cell from the relationship between the current and voltage applied to the electrode unit, or to acquire the electric resistance by outputting the current value and the voltage value to the control device described later in detail.

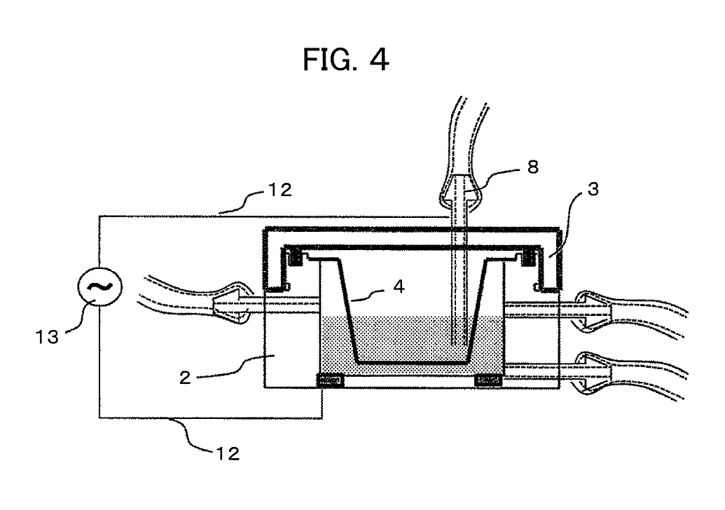

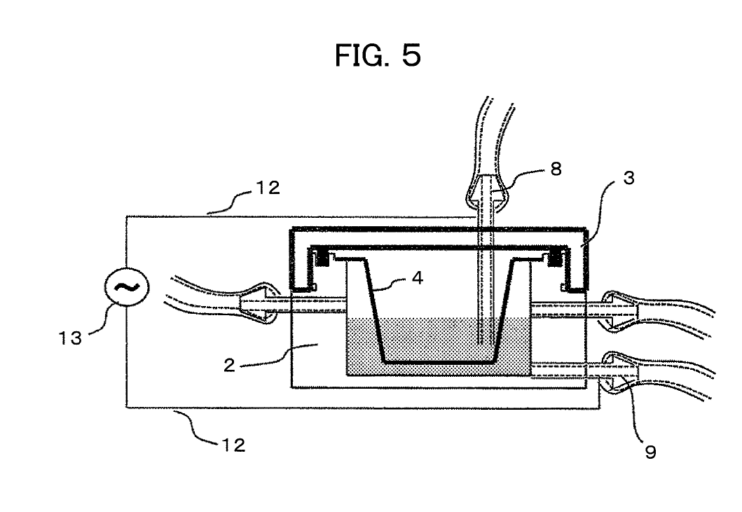

On the other hand, the flow channel 8 formed on the lid 3 is made of an electrically conductive material according to the modification illustrated in FIG. 4. With this structure, the connection to the AC voltage generating device 13 that can measure the electric resistance is made, and the electric wire is connected to the flow channel, whereby the electric resistance can be measured without providing another electrode. In this case, the cell culture container table 14 may be provided on the bottom of the frame body 2 as in FIG. 3. In the modification in FIG. 5, the flow channel 9 on the frame body 2 is made of an electrically conductive material in addition to the structure in FIG. 4, wherein the connection to the AC voltage generating device 13 serving as an electric resistance measuring device is made, whereby the electric resistance can similarly be measured.

In the modification in FIG. 6, a circular electrode 15 that is the second electrode as illustrated in the perspective view in the lower part of FIG. 6 is arranged on the side face of the culture holding face of the frame body 2, i.e., on the side face of the frame body, the rod electrode 11 is arranged on the lid 3 along the flow channel 8, and the electric wire 12 from the electrode 11 is arranged outside the frame body, and is connected to the AC voltage generating device 13 that can measure the electric resistance. The way of connecting the AC voltage generating device 13 and the cell culture container 1 may be the same as that described in FIG. 2.

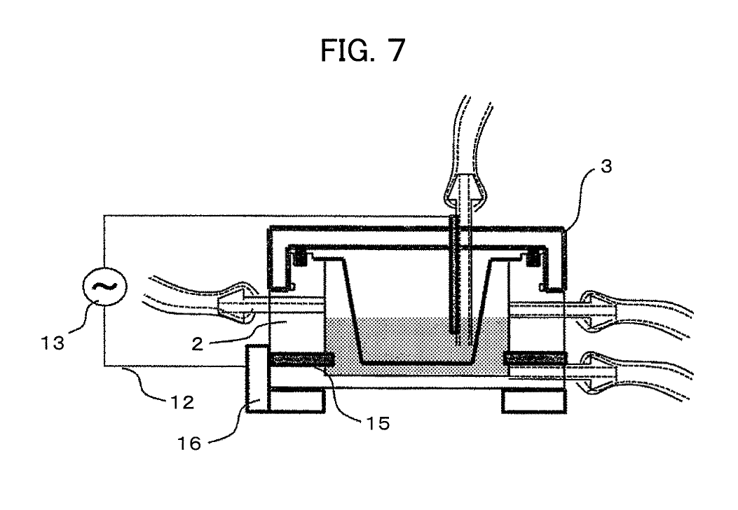

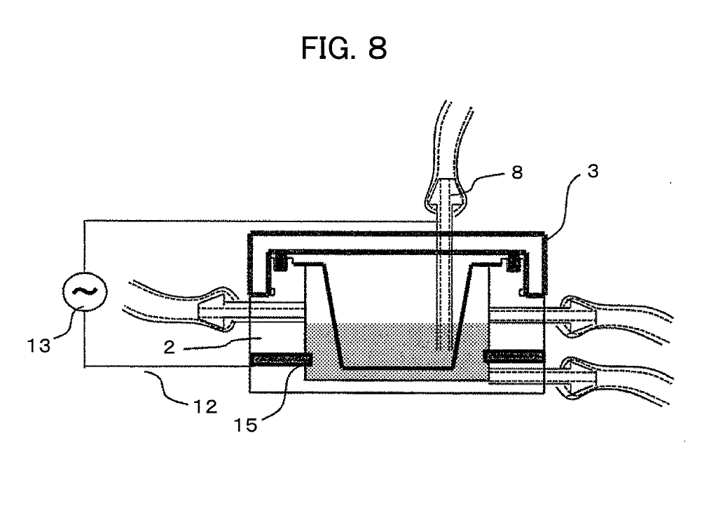

FIGS. 7 and 8 are another modification of the modification in FIG. 6. In FIG. 7, the electric wire is not directly connected to the electrode 15 on the side face of the frame body 2, but a cell culture container table 16 that can carry current to the electrode 15 through the connection to the electric wire 12 is provided. With this structure, only when the cell culture container 1 is placed on the cell culture container table 16, the electric resistance for the circular electrode 15 on the bottom can be measured. In FIG. 8, the flow channel 8 formed on the lid 3 is made of an electrically conductive material. With this structure, the electric resistance can be measured by connecting the electric wire 12 to the flow channel 8 without providing another electrode. In this case, the cell culture container table 16 may be mounted on the bottom of the frame body 2 as in FIG. 7.

### <Configuration of Cell Culturing Apparatus>

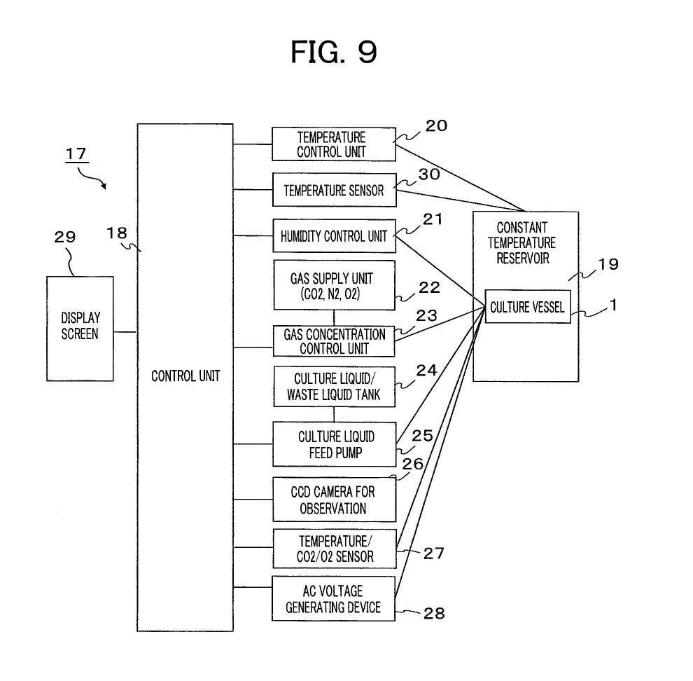

FIG. 9 is a block diagram for describing a functional configuration of a cell culturing apparatus 17 to which various cell culture containers 1 described above can be connected. FIG. 9 illustrates the entire configuration in which each component controlled by the control device 18 is connected to the cell culture container 1 arranged in a constant temperature reservoir 19. It is obvious that the components arranged in the constant temperature reservoir 19 is the culture container having the closed structure, or the culture container having the closed structure placed on the cell culture container table, described with reference to FIGS. 2 to 8.

Connected to the control device 18 are a temperature control unit 20 for controlling the temperature in the constant temperature reservoir 19, a humidity control unit 21 for controlling humidity in the culture container, a gas concentration control unit 23 having a gas supply unit 22 for controlling a concentration of gas in the culture container, a culture liquid feed pump 25 that has a liquid feed tube connected to a tank 24, holding the culture liquid and waste liquid, for automatically exchanging the culture liquid in the culture container, a CCD (Charge Coupled Device) camera 26 for the observation of the cell for a purpose of controlling the operation of each component, a temperature/humidity/CO2/O2 sensor 27, an AC voltage generating device 28 for measuring transepithelial electrical resistance, and a device for displaying a display screen 29. The control device 18 can acquire the electric resistance value of the cultured cell by receiving the electric resistance value or current/voltage value from the AC voltage generating device 28.

The control device 18 and the display screen 29 correspond to a processing unit and a storage unit, and a display unit of a display device of a general computer provided with a processing unit and a storage unit, which are composed of a central processing unit (CPU) and an input/output unit including a display device and a keyboard. The control device 18 runs various programs stored in the storage unit on the CPU serving as the processing unit to control the components ranging from the temperature control unit 20 to the AC voltage generating device 28. With this, the control device 18 can control the culture environment in the constant temperature reservoir 19, thereby enabling a prescribed culture in the culture container 1.

The humidity control unit 21 and the gas concentration control unit 23 do not have to be directly connected to the culture container 1. The temperature control unit 20, the humidity control unit 21, the gas concentration control unit 23, and the temperature/humidity/CO2/O2 sensor 27 may be connected to the constant temperature reservoir 19. In this configuration, the gas has to be supplied to the cell culture container 1 from the outside of the container. Therefore, a transparent thin film having gas permeability made of polycarbonate, polystyrene, or polymethylpentene is deposited on a part of the lid 3 of the cell culture container 1 in order to enable the gas exchange in the cell culture container 1, whereby the cell culture can be executed.

### <Culture Continuation Determination Program>

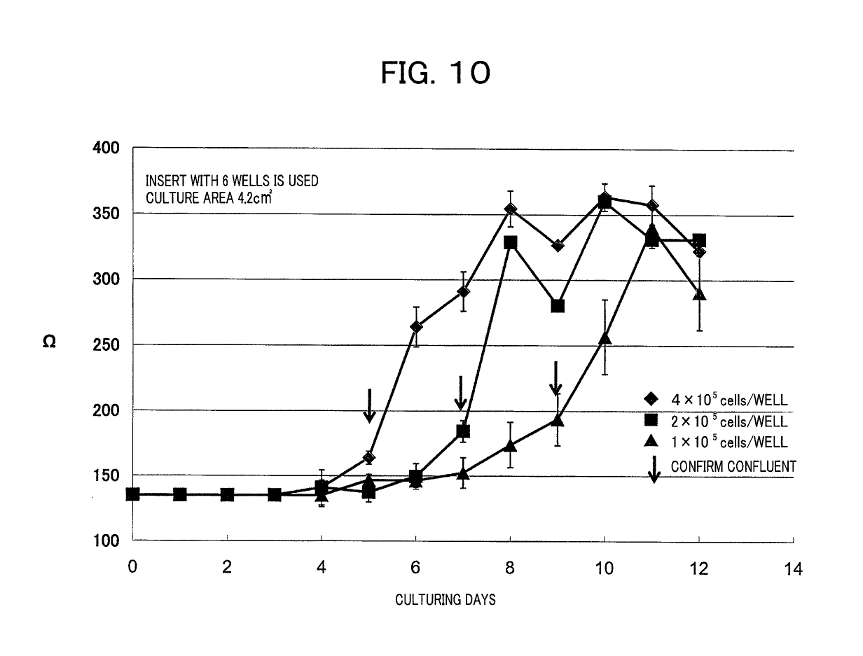

FIG. 10 is a view illustrating an electric resistance value measured by using an existing electric resistance measuring device, when corneal epithelial cell of a rabbit is cultured in the insert container 4 in the cell culture container 1, on which the electrode described above is arranged, according to the present example. As illustrated in FIG. 10, the cell to be seeded is cultured in three patterns, and arrows indicate the time when the cells spread all over the culture face to become confluent. It is found from this figure that the electric resistance value increases more than the background on the time when the cells become confluent, i.e., the time of starting the formation of the occluding junction. The continuation or discontinuation of the culture can be determined by the cell observation and by utilizing the change in the resistance value described above during the automatic culture.

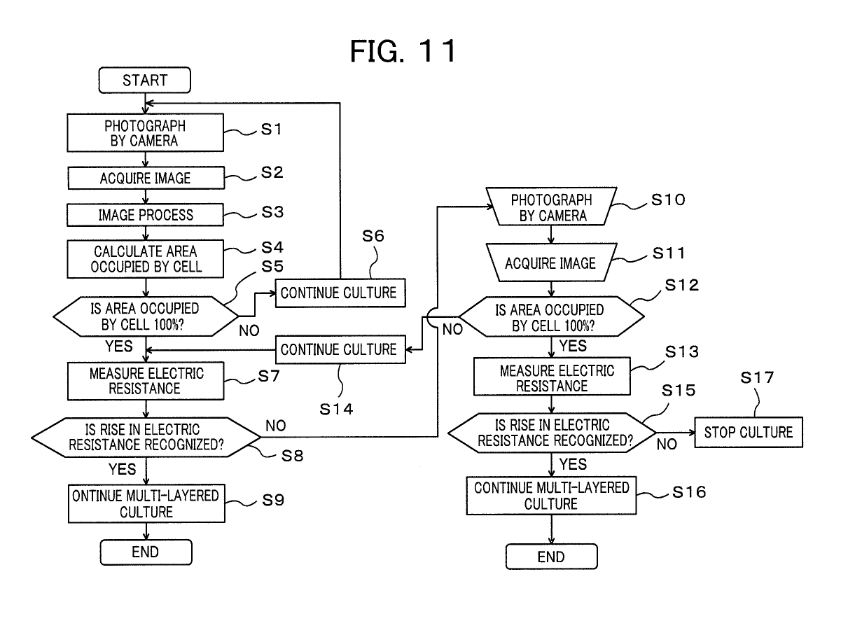

FIG. 11 illustrates one example of a culture continuation determination program of the cell culturing apparatus 17 according to the present example. This program is executed by the control device 18 including the CPU and the storage unit. With the start of the automatic culture, the CCD camera 26 images the cell (step S1) to acquire a cell image (step S2). The control unit 18 described above runs a predetermined image processing program, thereby executing a process of detecting a cell from the acquired image data (step S3). The control unit 18 then calculates an area occupied by the cell in the image after the binarization (step S4). When the area occupied by the cell does not reach a predetermined area after the control unit 18 acquires some data on the culture face, the control unit 18 continues the culture without measuring the electric resistance value, and repeats the processes in steps S1 to S5 on a predetermined timing (step S6).

When the control device 18 determines that the area occupied by the cell is 100% from the determination result in S5, it controls to measure the electric resistance value by the AC voltage generating device 28 (step S7). When the rise in the electric resistance value acquired from the AC voltage generating device 18 is recognized, compared to the background, as a result of the measurement (Yes in step S8), a multi-layered culture is continued (step S9), and then, the culture is ended on a predetermined timing.

When the rise in the measured electric resistance value is not recognized (No in step S8), the culture is continued for a predetermined time. The camera is manually operated (step S10) to acquire an image (step S11), and the control unit determines whether the area occupied by the cell is 100% or not (step S12). When the area occupied by the cell does not reach 100%, the culture is continued (step S14), and the processes in steps S7 to S12 are repeated.

When the area occupied by the cell is 100% (Yes in step S12), the electric resistance value is measured (step S13) to determine whether the rise in the electric resistance value is recognized or not (step S15). When the rise in the electric resistance value is recognized, the multi-layered culture is continued (step S16), and the culture is ended on a predetermined timing. When the rise in the electric resistance value is not recognized, the culture is stopped (step S17).

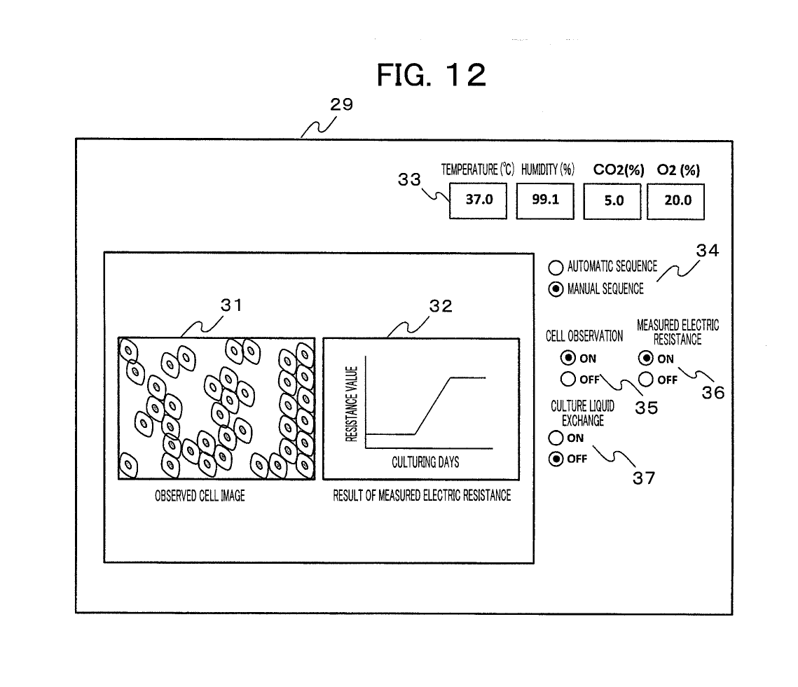

FIG. 12 illustrates one example of the display screen 29 on the display device during the execution of the culturing process by the cell culturing apparatus according to the present example. In FIG. 12, an observed cell image 31 and a result of measured electric resistance 32 are displayed on an image display area in the display screen 29. The display screen 29 also displays temperature, humidity, and concentration display area 33 indicating the environment temperature, humidity, CO2 concentration, and O2 concentration in the constant temperature reservoir 19. The display screen 29 also displays a button 34 for selecting an automatic sequence or manual sequence of the culturing process, buttons 35 and 36 for selecting a cell observation mode and an electric resistance measurement mode, and a button 37 for selecting a culture liquid exchange mode.

The present invention is not limited to the example described above. The epithelial cell is mainly described as the cell to be cultured. However, the cell to be cultured is not limited to the epithelial cell, and various cell species can be employed. The example described above is described in detail for better understanding of the present invention, and the present invention is not limited to the one including all components described above. Some components in the example can be replaced by the components of the other example, and the components in a certain example can be added to the components in the other example. Various additions, omissions, and replacements are possible for some components in each example.

A part or all of components, functions, and processing units described above may obviously be realized by specialized hardware designed by an integrated circuit. The information realizing each function can be stored not only in a memory serving as the storage unit, but also in a memory device such as hard disk or SSD (Solid State Drive) or a memory medium such as an IC (Integrated Circuit) card.

### INDUSTRIAL APPLICABILITY

The present invention is well adaptable to a cell culture container that realizes a non-invasive quality evaluation of a cell, and a cell culturing apparatus using the cell culture container.

### REFERENCE SIGNS LIST

1 . . . cell culture container,

2 . . . frame body,

3 . . . lid,

4 . . . insert container,

5 . . . elastic member,

6, 7, 8 . . . flow channel,

9 . . . tube,

10, 11, 15 . . . electrode,

12 . . . electric wire,

13 . . . AC voltage generating device,

14, 16 . . . cell culture container table,

17 . . . cell culturing apparatus,

18 . . . control device,

19 . . . constant temperature reservoir,

20 . . . temperature control unit,

21 . . . humidity control unit,

22 . . . gas supply unit,

23 . . . gas concentration control unit,

24 . . . culture liquid/waste liquid tank,

25 . . . culture liquid feed pump,

26 . . . CCD camera for observation,

27 . . . humidity/CO2/O2 sensor,

28 . . . AC voltage generating device,

29 . . . display screen,

30 . . . temperature sensor,

31 . . . observed cell image,

32 . . . result of measured electric resistance,

33 . . . temperature/humidity/concentration display area,

34, 35, 36, 37 . . . button

1. A cell culture container for holding and culturing a cell, the cell culture container comprising:
a frame body that holds culture liquid for culturing the cell;
a lid that is detachably mounted on the frame body;
a first electrode mounted on a bottom or on a side face of the frame body and having a shape enabling a cell observation, and
a second electrode mounted on the lid and having a shape enabling a cell observation.

2. The cell culture container according to claim 1,
wherein the first electrode is composed of a circular electrode formed on the bottom of the frame body, and
the second electrode is composed of a rod electrode formed on the lid.

3. The cell culture container according to claim 1,
wherein the lid is formed with a flow channel from which the culture liquid is introduced to the frame body,
the first electrode is composed of a circular electrode formed on the bottom of the frame body, and
the second electrode is configured to be capable of energizing the flow channel formed on the lid.

4. The cell culture container according to claim 1, wherein the first electrode is composed of a circular electrode formed on the side face of the frame body.

5. The cell culture container according to claim 4,
wherein the lid is formed with a flow channel from which the culture liquid is introduced into the frame body, and
the second electrode is configured to be capable of energizing the flow channel formed on the lid.

6. A cell culturing apparatus that measures an electric resistance of a cell in a cell culture container, the cell culturing apparatus comprising:
a closed culture container for culturing a cell;
a constant temperature reservoir for culturing a cell in which the closed culture container is placed;
a control device that controls a culture environment in the closed culture container; and
an AC voltage generating device,
wherein the closed culture container includes a frame body that holds culture liquid for culturing the cell; a lid that is detachably mounted on the frame body; a first electrode mounted on a bottom or on a side face of the frame body and having a shape enabling a cell observation; and a second electrode mounted on the lid and having a shape enabling a cell observation, and
the AC voltage generating device applies an AC voltage between the first electrode and the second electrode during the culture of the cell in the closed culture container.

7. The cell culturing apparatus according to claim 6,
wherein the first electrode is composed of a circular electrode formed on the bottom of the frame body, and
the second electrode is composed of a rod electrode formed on the lid.

8. The cell culturing apparatus according to claim 6,
wherein the lid is formed with a flow channel from which the culture liquid is introduced to the frame body,
the first electrode is composed of a circular electrode formed on the bottom of the frame body, and
the second electrode is configured to be capable of energizing the flow channel formed on the lid.

9. The cell culturing apparatus according to claim 6, wherein the first electrode is composed of a circular electrode formed on the side face of the frame body.

10. The cell culturing apparatus according to claim 9,
wherein the lid is formed with a flow channel from which the culture liquid is introduced into the frame body, and
the second electrode is configured to be capable of energizing the flow channel formed on the lid.

11. The cell culturing apparatus according to claim 7, further comprising:
a cell culture container table that is connected to the AC voltage generating device and that can be energized,
wherein the cell culture container table on which the closed culture container is placed is mounted in the constant temperature reservoir for culturing a cell.

12. The cell culturing apparatus according to claim 8, further comprising:
a cell culture container table that is connected to the AC voltage generating device and that can be energized,
wherein the cell culture container table on which the closed culture container is placed is mounted in the constant temperature reservoir for culturing a cell.

13. The cell culturing apparatus according to claim 9, further comprising:
a cell culture container table that is connected to the AC voltage generating device and that can be energized,
wherein the cell culture container table on which the closed culture container is placed is mounted in the constant temperature reservoir for culturing a cell.

14. The cell culturing apparatus according to claim 6, wherein the control device determines a continuation or discontinuation of the culture based upon a measurement result of electric resistance by the AC voltage generating device.

15. The cell culturing apparatus according to claim 6, wherein the control device controls a feed of a gas to the closed culture container in order to control the gas concentration in the closed culture container placed in the constant temperature reservoir for culturing a cell.
